(19) United States
(12) Patent Application Publication
(10) Pub. No.: US 2004/0067566 A1
(43) Pub. Date: Apr. 8, 2004

(54) Process for producing coenzyme q10

(76) Inventors: Hideyuki Matsuda, Matsue-shi (JP); Makoto Kawamukai, Matsue-shi (JP); Kazuyoshi Yajima, Akashi-shi (JP); Yasuhiro Ikenaka, Kobe-shi (JP)

Correspondence Address: KENYON & KENYON, 1500 K STREET, N.W., SUITE 700, WASHINGTON, DC, 20005, US

(21) Appl. No.: 10/416,855

(22) PCT Filed: Nov. 20, 2001
(86) PCT No.: PCT/JP01/10119

(30) Foreign Application Priority Data
Nov. 20, 2000 (JP) 2000-352940

(51) Int. Cl. C12P 7/26; C07H 21/04; C12N 9/10; C12N 1/16; C12N 15/74

(52) U.S. Cl. 435/148; 435/69.1; 435/193; 435/320.1; 435/254.1; 536/23.2

(57) ABSTRACT

The invention aims at providing a process for producing coenzyme Q₁₀ efficiently in microorganisms by utilizing a coenzyme Q₁₀ side chain synthesis gene derived from a fungal species belonging to the genus Rhodotorula.

The present invention relates to a DNA having a DNA sequence described under SEQ ID NO:1, 3 or 5 or derived from the above sequence by deletion, addition, insertion and/or substitution of one or several bases and encoding a protein having decaprenyl diphosphate synthase activity.

### TECHNICAL FIELD

The present invention relates to a process for producing coenzyme Q₁₀, which is in use as a drug or the like. More particularly, it relates to a process for causing formation of coenzyme Q₁₀ by isolating a gene coding for a coenzyme Q₁₀ side chain synthase, which serves as a key enzyme in the biosynthesis of coenzyme Q₁₀, namely a decaprenyl diphosphate synthase, from a fungal species belonging to the genus Rhodotorula and introducing that gene into a host microorganism.

### BACKGROUND ART

An industrial process for producing coenzyme Q₁₀, which is conventional in the art, comprises, for example, isolating coenzymes Q of plant origin, for example of tobacco origin, and adjusting the side chain length thereof by a synthetic method.

It is known that coenzyme Q₁₀ is produced in a wide variety of organisms, from microorganisms, such as bacteria and yeasts, to higher animals and plants. Thus, the process comprising cultivating a microorganism and extracting this substance from cells thereof can be regarded as one of the most efficient process for producing Q₁₀ and has actually been employed in commercial production thereof. However, the productivity of such processes can hardly be said to be good, since the yield is low and the procedure is complicated, for instance.

As for analogs (e.g. coenzyme Q₈) differing in chain length from coenzyme Q₁₀, not Q₁₀ itself, attempts have also been made to increase the production thereof by isolating genes involved in the biosynthesis thereof and amplifying the genes utilizing the recombinant DNA technology.

Coenzyme Q₁₀ is formed in vivo in a multistage process comprising complicated reactions in which a number of enzymes are involved. The route of biosynthesis thereof in prokaryotes partially differs from that in eukaryotes. Basically, however, each route comprises three fundamental steps, namely the step of synthesis of decaprenyl diphosphate, which is the source of the prenyl side chain of coenzyme Q₁₀, the step of synthesis of para-hydroxybenzoic acid, which is the source of the quinone ring, and the step of completion of coenzyme Q₁₀ through coupling of these two compounds and successive substituent conversions. Among these reactions, the reaction which determines the side chain length of coenzyme Q₁₀, namely the decaprenyl diphosphate synthase-involving reaction, which is said to be a rate-determining one in the whole biosynthetic reaction route, is considered to be the most important one.

Therefore, for efficient production of coenzyme Q₁₀, it is considered effective to isolate a decaprenyl diphosphate synthase gene, which is the key gene in the biosynthesis of coenzyme Q₁₀, and utilize the same for the purpose of increasing production. As for the gene source, fungi, in which coenzyme Q₁₀ is produced in relatively large amounts, are leading candidates.

So far, decaprenyl diphosphate synthase genes have been isolated from several microorganisms, such as Schizosa-charomyces pombe (JP-A-09-173076) and Gluconobacter suboxydans (JP-A-10-57072). However, the productivity of coenzyme Q₁₀ in these microorganisms cannot be said to be satisfactory, and the cultivation of these microorganisms and the separation/purification of coenzyme Q₁₀ therefrom have not been efficient. It has thus been desired that a microorganism-derived gene for that enzyme, which enables high level production of coenzyme Q₁₀, be isolated.

### SUMMARY OF THE INVENTION

It is an object of the present invention to produce coenzyme Q₁₀ efficiently in microorganisms by isolating a coenzyme Q₁₀ side chain synthesis gene from a fungal species belonging to the genus Rhodotorula and utilizing the same to hereby solve the above-mentioned productivity problem.

For attaining the above object, the present inventors made investigations in an attempt to isolate a decaprenyl diphosphate synthase gene from fungi belonging to the genus Rhodotorula, in which coenzyme Q₁₀ is produced in relatively large amounts, and succeeded in isolating such gene. As a result of further investigations made by them to increase the expression level of that gene, they succeeded in improving the gene so that coenzyme Q₁₀ may be expressed more abundantly. These successes have now led to completion of the present invention.

Thus, the present invention provides

a DNA of the following (a), (b) or (c):

(a) a DNA whose base sequence is as described under SEQ ID NO:1;

(b) a DNA having a DNA sequence derived from the base sequence shown under SEQ ID NO:1 by deletion, addition, insertion and/or substitution of one or several bases and

encoding a protein having decaprenyl diphosphate synthase activity;

(c) a DNA capable of hybridizing with a DNA comprising the base sequence shown under SEQ ID NO:1 under stringent conditions and

encoding a protein having decaprenyl diphosphate synthase activity.

The invention further provides a DNA improved in its expression in prokaryotes, namely

a DNA of the following (d), (e) or (f):

(d) a DNA whose base sequence is as described under SEQ ID NO:3;

(e) a DNA having a DNA sequence derived from the base sequence shown under SEQ ID NO:3 by deletion, addition, insertion and/or substitution of one or several bases and

encoding a protein having decaprenyl diphosphate synthase activity;

(f) a DNA capable of hybridizing with a DNA comprising the base sequence shown under SEQ ID NO:3 under stringent conditions and

encoding a protein having decaprenyl diphosphate synthase activity.

The invention further provides a DNA improved in its expression in prokaryotes, namely

a DNA of the following (k), (1) or (m):

(k) a DNA whose base sequence is as described under SEQ ID NO:5;

(l) a DNA having a DNA sequence derived from the base sequence shown under SEQ ID NO:5 by deletion, addition, insertion and/or substitution of one or several bases and

encoding a protein having decaprenyl diphosphate synthase activity;

(m) a DNA capable of hybridizing with a DNA comprising the base sequence shown under SEQ ID NO:5 under stringent conditions and

encoding a protein having decaprenyl diphosphate synthase activity.

The invention further provides

a protein of the following (g) or (h):

(g) a protein whose amino acid sequence is as described under SEQ ID NO:2;

(h) a protein having an amino acid sequence derived from the amino acid sequence shown under SEQ ID NO:2 by deletion, addition, insertion and/or substitution of one or several amino acid residues and having decaprenyl diphosphate synthase activity.

The invention further provides

a protein of the following (i) or (j):

(i) a protein whose amino acid sequence is as described under SEQ ID NO:4;

(j) a protein having an amino acid sequence derived from the amino acid sequence shown under SEQ ID NO:4 by deletion, addition, insertion and/or substitution of one or several amino acid residues and having decaprenyl diphosphate synthase activity.

The invention further provides

a protein of the following (n) or (o):

(n) a protein whose amino acid sequence is as described under SEQ ID NO:6;

(o) a protein having an amino acid sequence derived from the amino acid sequence shown under SEQ ID NO:6 by deletion, addition, insertion and/or substitution of one or several amino acid residues and having decaprenyl diphosphate synthase activity.

The invention further provides

DNAs respectively encoding the above proteins (g) to

The invention further provides

expression vectors with the above DNAs inserted into vectors.

The invention further provides

transformants resulting from transformation of host microorganisms with the respective DNAs mentioned above or with the expression vectors mentioned above.

The invention further provides

a DNA encoding the protein (n) or (o).

The invention further provides

expression vectors with the above DNA inserted into vectors.

The invention further provides

transformants resulting from transformation of host microorganisms with the respective DNAs mentioned above or with the expression vectors mentioned above.

The invention still further provides

a process for producing coenzyme Q₁₀,

which comprises cultivating any of the above-mentioned transformants in a medium and recovering coenzyme Q₁₀ thus formed and accumulated in the medium.

### DETAILED DISCLOSURE OF THE INVENTION

In the following, the present invention is described in detail.

The present inventors made investigations to isolate a gene encoding the enzyme in question from fungi belonging to the genus Rhodotorula, in which coenzyme Q₁₀ is produced in relatively large amounts, and, as a result, succeeded in obtaining a fragment of the gene by the PCR method.

The sequence of a known gene encoding a decaprenyl diphosphate synthase was compared with that of a known gene encoding a polyprenyl diphosphate synthase which is an analogue with different chain length of the enzyme in question and a long prenyl chain synthase of coenzyme Q, to the decaprenyl diphosphate synthase, and various PCR primers were synthesized for regions showing high homology therebetween. PCR conditions were studied for various combinations of these primers and, as a result, it was revealed, by gene base sequence analysis, that when 40 PCR cycles, each comprising 94° C., 1 minute→43° C., 2 minutes→72° C., 2 minutes, are carried out after 3 minutes of heat treatment at 94° C., using the primers DPS-1 (5′-AAGGATCCTNYTNCAYGAYGAYGT-3′) and DPS-1 1AS (5′-ARYTGNADRAAYTCNCC-3′) (in these sequences, R representing A or G, Y representing C or T, and N representing G, A, T or C), a fragment, about 220 bp in size, of the enzyme gene in question is amplified from the chromosomal gene of Rhodotorula minuta IFO 0387, which is a fungal species belonging to the genus Rhodotorula.

For obtaining the enzyme gene in its full length, the chromosomal gene of Rhodotorula minuta IFO 0387 was cleaved with the restriction enzyme EcoRI, and the cleavage products were inserted into a λ phage vector to construct a recombinant phage library. The resulting plaques were transferred to a nylon membrane, and plaque hybridization was carried out using the PCR fragment in a labeled form, whereby a clone having the decaprenyl diphosphate synthase gene in its full length could be obtained.

The base sequence of the decaprenyl diphosphate synthase gene contained in the clone obtained was determined, whereupon it was revealed that it has the sequence shown under SEQ ID NO:1 in the sequence listing. In the amino acid sequence (amino acid sequence described under SEQ ID NO:2 in the sequence listing) predicted from this base sequence, there was found a sequence characteristic of decaprenyl diphosphate synthase.

Since, in eukaryotes, the decaprenyl diphosphate synthase gene is expressed and functions in the mitochondria, it is supposed that, in the sequence on the amino acid terminal side of this gene sequence, there be a sequence allowing localization thereof in the mitochondria. Therefore, the inventors considered that, for more effective functioning of this gene in prokaryotes, it be necessary to specify and eliminate that or those sequences which is or are not essential in prokaryotes. As a result of studies on the amino acid terminal side sequence of that gene sequence, it has become possible to produce coenzyme Q₁₀ in significant amounts by using the gene specified under SEQ ID NO:3. The amino acid sequence deducible from the DNA sequence shown under SEQ ID NO:3 is described under SEQ ID NO:4 in the sequence listing. As a result of further studies on the amino acid terminal side sequence, it has become possible to produce coenzyme Q₁₀ in significant amounts by using the gene specified under SEQ ID NO:5. The amino acid sequence deducible from the DNA sequence shown under SEQ ID NO:5 is described under SEQ ID NO:6 in the sequence listing.

The DNA of the present invention may be a DNA whose base sequence is as described under SEQ ID NO:1 or SEQ ID NO:3, or a DNA having a base sequence derived from the base sequence shown under SEQ ID NO:1 or SEQ ID NO:3 by deletion, addition, insertion and/or substitution of one or several bases and encoding a protein having decaprenyl diphosphate synthase activity, or a DNA capable of hybridizing with a DNA comprising the base sequence shown under SEQ ID NO:1 or SEQ ID NO:3 under stringent conditions and encoding a protein having decaprenyl diphosphate synthase activity. A number of amino acids each may be encoded by one or more codons (genetic code degeneracy), so that a number of DNAs other than the DNA having the base sequence shown under SEQ ID NO:1 or SEQ ID NO:3 can encode the protein having the amino acid sequence shown under SEQ ID NO:2 or SEQ ID NO:4. Therefore, the DNA of the invention includes such DNAs en-coding the protein having the amino acid sequence shown under SEQ ID NO:2 or SEQ ID NO:4 as well.

The DNA of the present invention may be a DNA whose base sequence is as described under SEQ ID NO:5, or a DNA having a base sequence derived from the base sequence shown under SEQ ID NO:5 by deletion, addition, insertion and/or substitution of one or several bases and encoding a protein having decaprenyl diphosphate synthase activity, or a DNA capable of hybridizing with a DNA comprising the base sequence shown under SEQ ID NO:5 under stringent conditions and encoding a protein having decaprenyl diphosphate synthase activity. A number of amino acids each may be encoded by one or more codons (genetic code degeneracy), so that a number of DNAs other than the DNA having the base sequence shown under SEQ ID NO:5 can encode the protein having the amino acid sequence shown under SEQ ID NO:6. Therefore, the DNA of the invention includes such DNAs encoding the protein having the amino acid sequence shown under SEQ ID NO:6 as well.

The expression “base sequence derived by deletion, addition, insertion and/or substitution of one or several bases” as used herein means a base sequence resulting from deletion, addition, insertion and/or substitution of such a number of bases as can be deleted, added, inserted and/or substituted according to the methods well known to those skilled in the art, for example those described in Supplemental issue, Tanpakushitsu, Kakusan, Koso (Protein, Nucleic Acid and Enzyme), PCR Method for Gene Amplification, TAKKAJ, 35 (17), 2951-3178 (1990) or Henry A. Erlich (ed.), translated into Japanese under the supervision of Ikunoshin Kato: PCR Technology (1990).

The expression “DNA capable of hybridizing with a DNA comprising the base sequence shown under SEQ ID NO:1 (or SEQ ID NO:3 or SEQ ID NO:5) under stringent conditions” means a DNA obtainable by utilizing the technique of colony hybridization, plaque hybridization or southern hybridization, among others, using a DNA comprising the base sequence shown under SEQ ID NO:1 (or SEQ ID NO:3 or SEQ ID NO:5) as a probe. Those skilled in the art would be able to readily obtain the desired DNA by carrying out such hybridization according to the method described in Molecular Cloning, 2nd edition (Cold Spring Harbor Laboratry Press, 1989).

The expression “protein having decaprenyl diphosphate synthase activity” means a protein capable of synthesizing decaprenyl diphosphate in a yield of not less than 10%, preferably not less than 40%, more preferably not less than 60%, still more preferably not less than 80%, as compared with the case where a protein having the amino acid sequence shown under SEQ ID NO:2, SEQ ID NO:4 or SEQ ID NO:6. Such capacity can be measured by reacting FPP (farnesyl diphosphate) with ¹⁴C-IPP (radiolabeled isopentenyl diphosphate) in the presence of the enzyme in question, hydrolyzing the resulting ¹⁴C-DPP (decaprenyl diphosphate) with phosphatase and, after separation by TLC, determining the incorporation in each spot for each chain length (Okada et al., Eur. J. Biochem., 255, 52-59).

The protein of the invention may be a protein whose amino acid sequence is as described under SEQ ID NO:2, SEQ ID NO:4 or SEQ ID NO:6, or a protein having an amino acid sequence derived from the amino acid sequence shown under SEQ ID NO:2, SEQ ID NO:4 or SEQ ID NO:6 by deletion, addition, insertion and/or substitution of one or several amino acid residues and having decaprenyl diphosphate synthase activity. “Such an amino acid sequence derived by deletion, addition, insertion and/or substitution of one or several amino acid residues” can be obtained by deleting, adding, inserting and/or substituting an amino acid residue or residues by site-specific mutagenesis or any other methods well known in the art. Such methods are specifically described, for example, in Nucleic Acid Res., 10, 6487 (1982) and Methods in Enzymology, 100, 448 (1983).

For causing expression of the decaprenyl diphosphate synthase gene, it is necessary to connect that gene to a site downstream of an appropriate promoter. It is possible to construct an expression vector, for example, by excising a DNA fragment containing the gene using a restriction enzyme or amplifying an enzyme-encoding gene portion alone by PCR and then inserting the fragment or amplification product into a promoter-containing vector.

In the practice of the invention, the vector in which a DNA encoding a protein having decaprenyl diphosphate synthase activity is to be inserted to give an expression vector is not particularly restricted but may be one derived from an Escherichia coli-derived plasmid with an appropriate promoter inserted therein. The Escherichia coli-derived plasmid includes, among others, pBR322, pBR325, pUC19, and pUC119, and the promoter includes, among others, the T7 promoter, trp promoter, tac promoter, lac promoter, and λPL promoter. In the practice of the invention, pGEX-2T, PGEX-3T, pGEX-3X (the three being products of Pharmacia), pBluescriptII, pUC19, pUC18 (product of Toyobo Co., Ltd.), pMALC2, pET-3T, pUCNT (described in WO 94/03613) and the like may also be used as vectors for expression. Among these, PUCNT is judiciously used. In specific examples, a decaprenyl diphosphate synthase gene expression vector, pNTRm2, can be constructed by inserting a DNA comprising the base sequence shown under SEQ ID NO:1 into the vector for expression pUCNT, and an expression vector, pNTRm6, can be constructed by inserting a DNA comprising the base sequence shown under SEQ ID NO:3 into PUCNT. An expression vector, pUCRm3, can be constructed when a DNA comprising the base sequence shown under SEQ ID NO:5 is inserted into the vector for expression pUC18.

And, by introducing the above enzyme gene expression vector into an appropriate microorganism, it becomes possible to utilize the microorganism for the production of coenzyme Q₁₀. The host microorganism is not particularly restricted but Escherichia coli is judiciously used. The Escherichia coli strain is not particularly restricted but includes XL1-Blue, BL-21, JM109, NM522, DH5α, HB101, DH5, and pUC18, among others. Among these, Escherichia coli HB101 and pUC18 are judiciously used. For example, when the decaprenyl diphosphate synthase gene expression vector pNTRm2, pNTRm6 or pUCRm3 is introduced into Escherichia coli, this can be transformed so that coenzyme Q₁₀, which Escherichia coli originally does not produce, can be produced in significant amounts. The Escherichia coli strain with pNTRm2 introduced therein has been deposited, under the Budapest Treaty, with the National Institute of Advanced Industrial Science and Technology International Patent Organism Depositary (Central 6, 1-1-1 Higashi, Tsukuba, Ibaraki, Japan) under the designation E. coli HB101(pNTRm2) FERM BP-15 7333, the Escherichia coli strain with pNTRm6 introduced therein under E. coli HB101(pNTRm6) FERM BP-7332, and the Escherichia coli strain with pUCRm3 under E. coli DH5α (pUCRm3) FERM BP-7638.

The gene of the invention may be used singly, or may be introduced into a microorganism together with another gene or other genes involved in the biosynthesis of coenzyme Q₁₀ for expression thereof. In the latter case, better results can be expected.

Coenzyme Q₁₀ can be produced by cultivating the transformant obtained in the invention in a medium in the conventional manner and recovering coenzyme Q₁₀ from the cultivation product. In cases where the host microorganism is Escherichia coli, LB medium, or M9 medium containing glucose and casamino acids can be used as the medium. For better promoter functioning, such an agent as isopropylthiogalactoside or indolyl-3-acrylic acid, for instance, may be added to the medium. The cultivation is carried out, for example, at 37° C. for 17 to 24 hours, if necessary with aeration and/or agitation. In the practice of the invention, the product coenzyme Q₁₀ obtained may be purified or used in the form of a crude product according to the selection duly made depending on the intended use thereof. Coenzyme Q₁₀ can be isolated from the ultivation product by an appropriate combination of per se known methods of separation and/or purification. The per se known methods of separation and/or purification include salting out, solvent precipitation and other methods utilizing the difference in solubility, dialysis, ultrafiltration, gel filtration, (SDS-)polyacrylamide gel electrophoresis and other methods mainly utilizing the difference in molecular weight, ion exchange chromatography and other methods utilizing the difference in charge, affinity chromatography and other methods utilizing specific affinity, reversed phase high-performance liquid chromatography and other methods utilizing the difference in hydrophobicity, isoelectric focusing and other methods utilizing the difference in isoelectric point, among others.

The field of utilization of coenzyme Q₁₀ obtained in the present invention is not particularly restricted but it may be judiciously used as a drug, among others.

### BRIEF DESCRIPTION OF THE DRAWINGS

FIG. 1 is a restriction enzyme map of the expression vector pNTRm2.

FIG. 2 is a restriction enzyme map of the expression vector pNTRm6.

FIG. 3 shows HPLC analysis charts for the host and transformant products.

FIG. 4 is a restriction enzyme map of the expression vector pUCRm3.

FIG. 5 shows HPLC analysis charts for the products in the host for the expression vector pUCRm3 and in the transformant.

### BEST MODES FOR CARRYING OUT THE INVENTION

The following examples illustrate the present invention in more detail. These examples are, however, by no means limitative of the scope of the invention.

### EXAMPLE 1

The chromosomal DNA of Rhodotorula minuta IFO 0387 was prepared by the method of C. S. Hoffman et al. (Gene, 57 (1987), 267-272). Based on the homology with the known long-chain prenyl diphosphate synthase gene, primers for use in PCR, namely DPS-1 (5′-AAGGATCCTNYTNCAYGAYGAYGT-3′) and DPS-1 1AS (5′-ARYTGNADRAAYTCNCC-3′), were designed. In these sequences, R represents A or G, Y represents C or T, and N represents G, A, T or C. Using these, a PCR cycle of 94° C., 1 minute→43° C., 2 minutes→72° C., 2 minutes, were repeated 40 times after 3 minutes of heat treatment at 94° C. (ExTaq, product of Takara, being used as the enzyme), followed by 1.2% agarose gel electrophoresis.

The thus-obtained fragment, about 220 bp in size, was purified by excising the corresponding gel portion from the gel and then treating with a DNA extraction kit (Sephaglas (trademark) BrandPrep Kit, product of Amersham Pharmacia Biotech), and the purified fragment was cloned into a vector for expression in Escherichia coli using a PCR product direct cloning kit (pT7 BlueT-Vector Kit, product of NOVAGEN) to give pT7-RmDPS. The DNA base sequence was determined by carrying out the reaction on a DNA sequencer (model 377, product of PerkinElmer) using a DNA sequencing kit (product of PerkinElmer, ABI PRISM (trademark) BigDye (trademark) Terminator Cycle Sequence Ready Reaction Kit with AmptiTaq (registered trademark) DNA polymerase, FS) and according to the manual attached thereto. As a result, a sequence covering the 823rd to 1029th bases of the base sequence shown under SEQ ID NO:1 in the sequence listing was obtained. The sequence “GDFLLARA”, which is a characteristic region of the long-chain prenyl chain-containing prenyl diphosphate synthase, could be found in the sequence translated from the base sequence determined in the above manner. It was therefore estimated that the sequence obtained be part of the decaprenyl diphosphate synthase gene.

### EXAMPLE 2

Using 0.03 μg of the pT7-RmDPS vector DNA having the 220 bp DNA fragment supposed to be part of the decaprenyl diphosphate synthase gene of Rhodotorula minuta IFO 0387 and using the PCR primers Rm-1S (having the sequence 5′-GCCATGAGGAGAGCACAAGCG-3′) and Rm-2AS (having the sequence 5′-CACGGAGGCTACTAGCTCGAC-3′), PCR (94° C., 3 minutes→(94° C., 30 seconds→55° C., 30 seconds→72° C., 1 minute)×25 cycles→72° C., 5 minutes→4° C.) was carried out, followed by 1.2% agarose (product of Takara Shuzo Co., Ltd.) gel electrophoresis. After excision of the corresponding portion from the gel, a fragment, about 145 bp in size, was purified using a DNA extraction kit (Sephaglas (trademark) BrandPrep Kit, product of Amersham Pharmacia Biotech). About 100 ng of this DNA fragment was subjected to chemiluminescence labeling using an ECL direct nucleic acid labeling system (product of Amersham Pharmacia Biotech).

### EXAMPLE 3

The chromosomal DNA of Rhodotorula minuta IFO 0387 was cleaved with the restriction enzyme EcoRI, followed by 0.8% agarose gel electrophoresis. The gel was denatured with an alkali (0.5 M NaOH, 1.5 M NaCl) and then neutralized (0.5 M Tris-HCl (pH 7.5), 1.5 M NaCl), a Hibond N+ filter (product of Amersham) was laid on the gel, and southern transfer was effected overnight using 20×SSC. The filter was dried and baked at 80° C. for 2 hours and, thereafter, southern hybridization and detection were carried out using an ECL direct nucleic acid labeling/detection system (product of Amersham Pharmacia Biotech). Thus, using a Gold hybridization solution (product of Amersham Pharmacia Biotech), prehybridization was carried out at 42° C. for 1 hour.

The chemiluminescence-labeled probe was heated at 95° C. for 5 minutes, then quenched in ice, and added to the prehybridization for the prehydridized filter, and hybridization was carried out at 42° C. for 22 hours. This filter was washed with two portions of a 0.5×SSC solution containing 6 M urea and 0.4% SDS at 42° C. for 20 minutes (for each portion) and then with two portions of a 2×SSC solution at room temperature for 5 minutes (for each portion). This filter was immersed in an enhanced chemiluminescence reagent (product of Amersham Pharmacia Biotech) and brought into close contact with an X ray film for exposure thereof, and black bands resulting from exposure were detected. As a result, strong hybridization with a fragment, about 5.5 kbp in size, resulting from the cleavage with the restriction enzyme EcoRI was detected.

### EXAMPLE 4

The chromosomal DNA of Rhodotorula minuta IFO 0387 was cleaved with the restriction enzyme EcoRI, followed by 0.8% agarose gel electrophoresis. A DNA fragment for use in cloning was prepared by excising the gel portion containing a DNA fragment in the vicinity of about 5.5 kbp in size and purifying the fragment. Using a λ-ZAPII phage kit (product of Stratagene), this DNA fragment was inserted into the phage at its EcoRI site, followed by packaging using an in vitro packaging kit (product of Amersham). Escherichia coli XL1-Blue MRF′ was infected with the phage and layered onto an NZY plate medium (5 g/L NaCl, 2 g/L MgSO₄.7H₂O, 5 g/L yeast extract, 10 g/L NZ amine, 18 g/L agar, pH 7.5), together with an NZY soft agar medium (same as the NZY plate medium except for the content of agar, which was 8 g/L), for plaque formation. The plaques were transferred onto a Hibond N+ filter (product of Amersham), denatured with an alkali (0.5 M NaOH, 1.5 M NaCl), neutralized (0.5 M Tris-HCl (pH 7.5), 1.5 M NaCl), dried, and baked at 80° C. for 2 hours.

Using 6 filters after baking as prepared in the above manner, prehybridization and hybridization using the chemiluminescence-labeled probe were carried out in the same manner as in Example 3, and the filters were washed. After drying, the filters were brought into close contact with an X ray film for exposure of the same, and the phage plaques corresponding to black spots resulting from exposure were isolated. Escherichia coli was infected with the phages of the thus-isolated plaques in the same manner as mentioned above for plaque formation. The plaques obtained were transferred onto a filter and again subjected to hybridization for confirmation. Seven phage strains could be selected.

Escherichia coli SOLR in the λ-ZAPII phage kit (product of Stratagene) was infected with each of those phages in a suspension form, together with the helper phage, and a phagemid was prepared in vitro. The above phagemid contained an insert fragment of about 5.5 kbp and, when PCR was carried out using the primers Rm-1S and Rm-2AS, the 145 bp DNA fragment could be detected. The DNA base sequence was determined by carrying out the reactions on a DNA sequencer (model 377, product of PerkinElmer, Inc.) using the internal primers Rm-1S and Rm-2AS and using a DNA sequencing kit (product of PerkinElmer, Inc., ABI PRISM (trademark) BigDye (trademark) Terminator Cycle Sequence Ready Reaction Kit with AmptiTaq (registered trademark) DNA polymerase, FS) according to the manual attached thereto. As a result of repeated sequencing using primers prepared based on the sequence revealed by preceding sequencing, the whole sequence of the decaprenyl diphosphate synthase gene of Rhodotorula minuta IFO 0387 could be revealed. Thus, the base sequence of a DNA of about 1.6 kbp was determined. The results are shown under SEQ ID NO:1 in the sequence listing. The amino acid sequence deduced from this DNA sequence is shown under SEQ ID NO:2.

### EXAMPLE 5

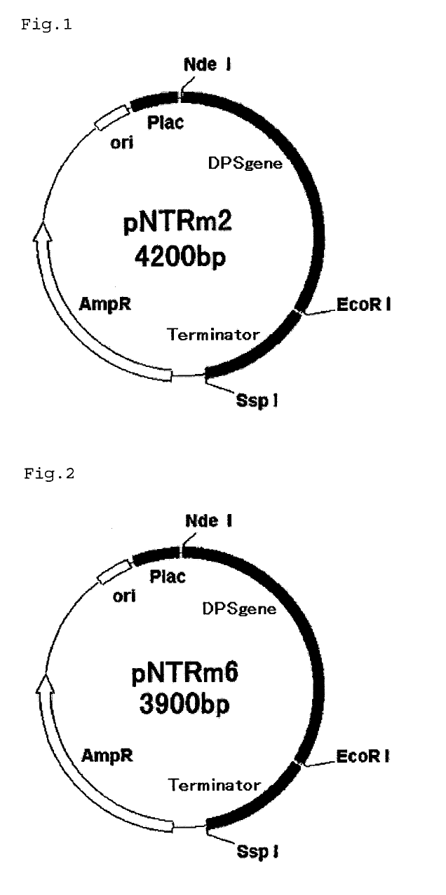

For excising the decaprenyl diphosphate synthase-encoding gene portion from the phagemid DNA prepared, PCR was carried out in the same manner as in Example 2 using two synthetic DNA primers designated RM-1 (having the sequence 5′-ATCATATGATGCACCGACAAGCT-3′) and Rm-CE2 (having the sequence 5′-AAGAATTCCTACTTTGTTCGGTTGAGCACAG-3′). The amplification product was cleaved with the restriction enzymes NdeI and EcoRI, and the cleavage product was inserted into a vector for expression, PUCNT (described in WO 94/03613) to give a decaprenyl diphosphate synthase gene expression vector, pNTRm2. The restriction enzyme map of the thus-obtained expression vector pNTRm2 is shown in FIG. 1. The symbol DPS stands for the coding region of the decaprenyl diphosphate synthase gene.

### EXAMPLE 6

The thus-constructed decaprenyl diphosphate synthase gene expression vector pNTRm2 was introduced into Escherichia coli HB101, the microorganisms were shake-cultured overnight in 10 mL of LB medium at 37° C., and cells were harvested by centrifugation (3,000 revolutions, 20 minutes).

The cells were suspended in 1 mL of a 3% aqueous solution of sulfuric acid and, after 30 minutes of heat treatment at 120° C., 2 mL of a 14% aqueous solution of sodium hydroxide was added, followed by further 15 minutes of heat treatment at 120° C. To the thus-treated suspension was added 3 mL of hexane-isopropanol (10:2) for effecting extraction. After centrifugation, 1.5 mL of the organic solvent layer was separated, and the solvent was evaporated to dryness under reduced pressure conditions. The residue was dissolved in 200 μl of ethanol, and 20 μl of the solution was analyzed by high-performance liquid chromatography (using LC-10A, product of Shimadzu Corp.). For separation, a reversed phase column (YMC-pack ODS-A, 250×4.6 mm, S-5 μm, 120A) was used, together with ethanol-methanol (2:1) as the mobile phase solvent. The coenzyme Q₁₀ formed was detected based on the absorbance at the wavelength 275 nm. The results are shown in FIG. 2. As shown in FIG. 2, it was revealed that, upon introduction of the decaprenyl diphosphate synthase gene for expression of Q₁₀, the transformant Escherichia coli strain was now possible to produce coenzyme Q₁₀, which is originally not produced in Escherichia coli.

The thus-obtained recombinant Escherichia coli HB101(pNTRm2) has been deposited, under the Budapest Treaty, with the National Institute of Advanced Industrial Science and Technology International Patent Organism Depositary (Central 6, 1-1-1 Higashi, Tsukuba, Ibaraki, Japan) as of Oct. 19, 2000 (deposition/accession No. FERM BP-7333).

### EXAMPLE 7

For eliminating the mitochondrial transitional sequence characteristic of eukaryotes from the sequence under SEQ ID NO:1 in order to attain abundant expression in prokaryotes, PCR was carried out in the same manner as in Example 2 using two synthetic DNA primers designated Rm-4 (having the sequence 5′-ATCATATGAATATTCGACCCACTCCAACT-3′) and Rm-CE2 (having the sequence 5′-AAGAATTCCTACTTTGTTCGGTTGAGCACAG-3′). The thus-amplified 1.3 kbp fragment was cleaved with the restriction enzymes NdeI and NheI, and the thus-prepared fragment, about 600 bp in size, was recombined to the major fragment of pNTRm2 as obtained by digestion with the restriction enzymes NdeI and NheI to construct pNTRmSsp. Further, PCR was carried out in the same manner as in Example 2 using two synthetic DNA primers designated RM-1 (having the sequence 5′-ATCATATGATGCACCGACAAGCT-3′) and RM-6R (having the sequence 5′-ACAATATTGTATTGAGGGCATTCGGGCGACTGC-3′) for amplifying a fragment, about 100 bp in size, resulting from deletion of the N portion. The fragment was cleaved with the restriction enzymes NdeI and SspI, and the resulting fragment was recombined to the major fragment of pNTRmSsp as resulting from digestion with the restriction enzymes NdeI and SspI to construct pNTRm6.

### EXAMPLE 8

The thus-constructed decaprenyl diphosphate synthase gene expression vector pNTRm6 was introduced into Escherichia coli HB101, the microorganisms were shake-cultured overnight in 10 mL of LB medium at 37° C., and cells were harvested by centrifugation (3,000 revolutions, 20 minutes).

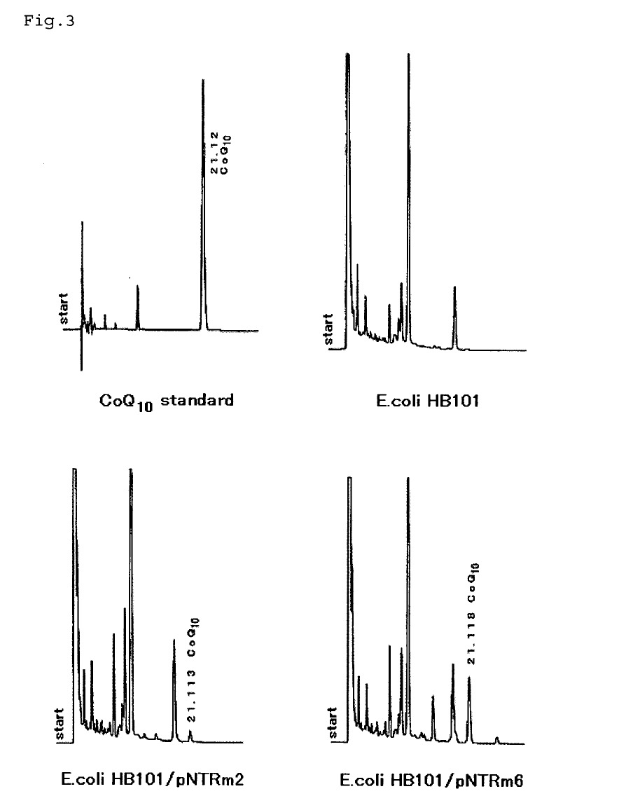

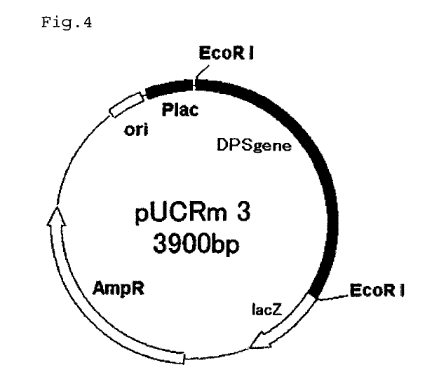

The cells were suspended in 1 mL of a 3% aqueous solution of sulfuric acid and, after 30 minutes of heat treatment at 120° C., 2 mL of a 14% aqueous solution of sodium hydroxide was added, followed by further 15 minutes of heat treatment at 120° C. To the thus-treated suspension was added 3 mL of hexane-isopropanol (10:2) for effecting extraction. After centrifugation, 1.5 mL of the organic solvent layer was separated, and the solvent was evaporated to dryness under reduced pressure conditions. The residue was dissolved in 200 μl of ethanol, and 20 μl of the solution was analyzed by high-performance liquid chromatography (using LC-10A, product of Shimadzu Corp.). For separation, a reversed phase column (YMC-pack ODS-A, 250×4.6 mm, S-5 μm, 120A) was used, together with ethanol-methanol (2:1) as the mobile phase solvent. The coenzyme Q₁₀ formed was detected based on the absorbance at the wavelength 275 nm. The results are shown in FIG. 3. As shown in FIG. 3, it was revealed that, upon introduction of the decaprenyl diphosphate synthase gene for expression thereof, coenzyme Q₁₀, which is originally not produced in Escherichia coli, could now be produced in the transformant and that conversion could be attained so as to attain coenzyme Q₁₀ production in significantly larger amounts as compared with the Escherichia coli strain transformed with pNTRm2.

The thus-obtained recombinant Escherichia coli HB101(pNTRm6) has been deposited, under the Budapest Treaty, with the National Institute of Advanced Industrial Science and Technology International Patent Organism Depositary (Central 6, 1-1-1 Higashi, Tsukuba, Ibaraki, Japan) as of Oct. 19, 2000 (deposition/accession No. FERM BP-7332).

### EXAMPLE 9

For eliminating the mitochondrial transitional sequence characteristic of eukaryotes from the sequence under SEQ ID NO:1 in order to attain abundant expression in prokaryotes, PCR was carried out in the same manner as in Example 2 using two synthetic DNA primers designated RmNEco (having the sequence 5′-ACGAATTCGATGATCTTCGACCCACTCCAACT-3′) and Rm-CE2 (having the sequence 5′-AAGAATTCCTACTTTGTTCGGTTGAGCACAG-3′), with pNTRm2 as the template. The thus-amplified 1.2 kbp fragment was cleaved with the restriction enzymes BamHI and EcoRI, and the thus prepared fragment was joined to the major fragment of pUC18 as obtained by digestion with the restriction enzymes BamHI and EcoRI to construct pUCRm3.

### EXAMPLE 10

The thus-constructed decaprenyl diphosphate synthase gene expression vector pUCRm3 was introduced into Escherichia coli DH5α, the microorganisms were shake-cultured overnight in 10 mL of LB medium at 37° C., and cells were harvested by centrifugation (3,000 revolutions, 20 minutes).

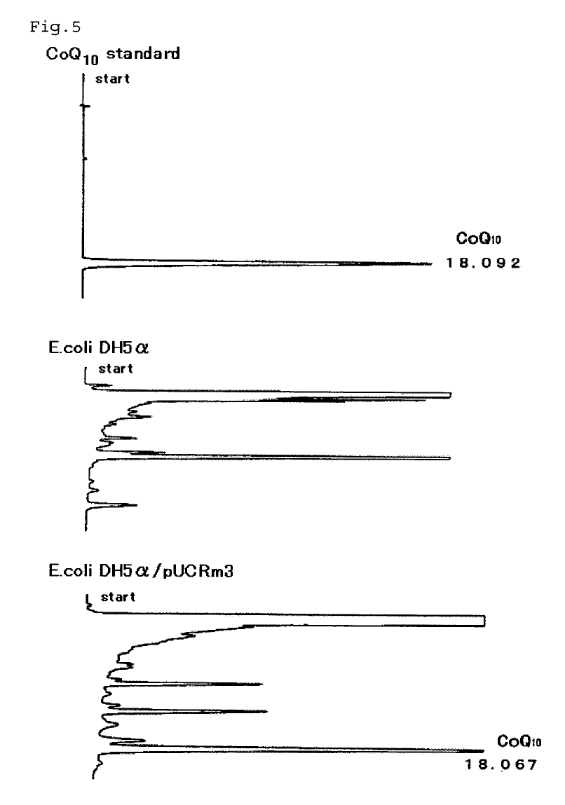

The cells were suspended in 1 mL of a 3% aqueous solution of sulfuric acid and, after 30 minutes of heat treatment at 120° C., 2 mL of a 14% aqueous solution of sodium hydroxide was added, followed by further 15 minutes of heat treatment at 120° C. To the thus-treated suspension was added 3 mL of hexane-isopropanol (10:2) for effecting extraction. After centrifugation, 1.5 mL of the organic solvent layer was separated, and the solvent was evaporated to dryness under reduced pressure conditions. The residue was dissolved in 200 μl of ethanol, and 20 μl of the solution was analyzed by high-performance liquid chromatography (using LC-10A, product of Shimadzu Corp.). For separation, a reversed phase column (YMC-pack ODS-A, 250×4.6 mm, S-5 μm, 120A) was used, together with ethanol-methanol (2:1) as the mobile phase solvent. The coenzyme Q₁₀ formed was detected based on the absorbance at the wavelength 275 nm. The results are shown in FIG. 3. As shown in FIG. 5, it was revealed that, upon introduction of the decaprenyl diphosphate synthase gene for expression thereof, coenzyme Q₁₀, which is originally not produced in Escherichia coli, could now be produced in the transformant and that conversion could be attained so as to attain coenzyme Q₁₀ production in significantly larger amounts as compared with the Escherichia coli strains transformed with pNTRm2 and pNTRm6, respectively.

The thus-obtained recombinant Escherichia coli DH5α (pUCRm3) has been deposited, under the Budapest Treaty, with the National Institute of Advanced Industrial Science and Technology International Patent Organism Depositary (Central 6, 1-1-1 Higashi, Tsukuba, Ibaraki, Japan) as of Jun. 22, 2001 (deposition/accession No. FERM BP-7638).

### Industrial Applicability

A gene encoding decaprenyl diphosphate synthase, which is the key enzyme in the biosynthesis of coenzyme Q₁₀, was isolated from a fungal species belonging to the genus Rhodotorula and it was sequenced. This could successfully be introduced in Escherichia coli for expression thereof. Furthermore, improvements in gene sequence successfully resulted in production of coenzyme Q₁₀ in significant amounts. By using the method of the invention, it becomes possible to efficiently produce coenzyme Q₁₀, which is in use as a drug, among others.

1. A DNA of the following (a), (b) or (c):
(a) a DNA whose base sequence is as described under SEQ ID NO:1;
(b) a DNA having a DNA sequence derived from the base sequence shown under SEQ ID NO:1 by deletion, addition, insertion and/or substitution of one or several bases and
encoding a protein having decaprenyl diphosphate synthase activity;
(c) a DNA capable of hybridizing with a DNA comprising the base sequence shown under SEQ ID NO:1 under stringent conditions and
encoding a protein having decaprenyl diphosphate synthase activity.

2. A DNA of the following (d), (e) or (f):
(d) a DNA whose base sequence is as described under SEQ ID NO:3;
(e) a DNA having a DNA sequence derived from the base sequence shown under SEQ ID NO:3 by deletion, addition, insertion and/or substitution of one or several bases and
encoding a protein having decaprenyl diphosphate synthase activity;
(f) a DNA capable of hybridizing with a DNA comprising the base sequence shown under SEQ ID NO:3 under stringent conditions and
encoding a protein having decaprenyl diphosphate synthase activity.

3. A protein of the following (g) or (h):
(g) a protein whose amino acid sequence is as described under SEQ ID NO:2;
(h) a protein having an amino acid sequence derived from acid sequence shown under SEQ ID NO:2 by deletion, insertion and/or substitution of one or several amino acid residues and having decaprenyl diphosphate synthase activity.

4. A protein of the following (i) or (j):
(i) a protein whose amino acid sequence is as described under SEQ ID NO:4;
(j) a protein having an amino acid sequence derived from the amino acid sequence shown under SEQ ID NO:4 by deletion, addition, insertion and/or substitution of one or several amino acid residues and having decaprenyl diphosphate synthase activity.

5. A DNA encoding the protein according to claim 3.

6. A DNA encoding the protein according to claim 4.

7. An expression vector resulting from insertion of the DNA according to claim 1, 2, 5 or 6 into a vector for expression.

8. The expression vector according to claim 7, wherein the vector for expression is pUCNT.

9. The expression vector according to claim 8, which is pNTRm2.

10. The expression vector according to claim 8, which is pNTRm6.

11. A transformant resulting from transformation of a host microorganism with the DNA according to claim 1, 2, 5 or 6.

12. A transformant resulting from transformation of a host microorganism with the expression vector according to claim 7, 8, 9 or 10.

13. The transformant according to claim 11 or 12,
wherein the host microorganism is a strain of Escherichia coli.

14. The transformant according to claim 13,
wherein the strain of Escherichia coli is Escherichia coli HB101.

15. The transformant according to claim 14,
which is E. coli HB101 (pNTRm2) (FERM BP-7333).

16. The transformant according to claim 14,
which is E. coli HB101 (pNTRm6) (FERM BP-7332).

17. A process for producing coenzyme Q₁₀,
which comprises cultivating the transformant according to claim 11, 12, 13, 14, 15 or 16 in a medium and recovering coenzyme Q₁₀ thus formed and accumulated in the medium.

18. A DNA of the following (k), (l) or (m):
(k) a DNA whose base sequence is as described under SEQ ID NO:5;
(l) a DNA having a DNA sequence derived from the base sequence shown under SEQ ID NO:5 by deletion, addition, insertion and/or substitution of one or several bases and
encoding a protein having decaprenyl diphosphate synthase activity;
(m) a DNA capable of hybridizing with a DNA comprising the base sequence shown under SEQ ID NO:5 under stringent conditions and
encoding a protein having decaprenyl diphosphate synthase activity.

19. A protein of the following (n) or (o):
(n) a protein whose amino acid sequence is as described under SEQ ID NO:6;
(o) a protein having an amino acid sequence derived from the amino acid sequence shown under SEQ ID NO:6 by deletion, addition, insertion and/or substitution of one or several amino acid residues and having decaprenyl diphosphate synthase activity.

20. A DNA encoding the protein according to claim 19.

21. An expression vector resulting from insertion of the DNA according to claim 18 or 20 into a vector for expression.

22. The expression vector according to claim 21,
wherein the vector for expression is pUC18.

23. The expression vector according to claim 22,
which is pUCRm3.

24. A transformant resulting from transformation of a host microorganism with the DNA according to claim 18 or 20.

25. A transformant resulting from transformation of a host microorganism with the expression vector according to claim 21, 22 or 23.

26. The transformant according to claim 24 or 25,
wherein the host microorganism is a strain of Escherichia coli.

27. The transformant according to claim 26,
wherein the strain of Escherichia coli is Escherichia coli DH5α.

28. The transformant according to claim 27,
which is E. coli DH5α (pUCRm3) (FERM BP-7638).

29. A process for producing coenzyme Q₁₀,
which comprises cultivating the transformant according to claim 24, 25, 26, 27 or 28 in a medium and recovering coenzyme Q₁₀ thus formed and accumulated in the medium.
